(19) United States
(12) Patent Application Publication
(10) Pub. No.: US 2014/0058219 A1
(43) Pub. Date: Feb. 27, 2014

(54) DEVICE FOR RECORDING ULTRASOUND-INDUCED FETAL EEG, AND METHOD OF USE

(71) Applicant: Marianna Kiraly, San Carlos, CA (US)

(72) Inventor: Marianna Kiraly, San Carlos, CA (US)

(21) Appl. No.: 14/068,951

(22) Filed: Oct. 31, 2013

Related U.S. Application Data
(63) Continuation-in-part of application No. 13/396,233, filed on Feb. 14, 2012.

(51) Int. Cl. A61B 5/0484; A61B 8/08; A61B 5/04; A61B 8/13; A61B 5/0478; A61B 8/02; A61B 5/0448; A61B 5/0205; A61B 8/00; A61B 5/00

(52) U.S. Cl. CPC A61B 5/0484; A61B 8/463; A61B 8/488; A61B 5/04017; A61B 5/4094; A61B 5/0478; A61B 5/7425; A61B 5/7203; A61B 8/02; A61B 8/0866; A61B 8/523; A61B 5/0448; A61B 5/0205; A61B 5/0006; A61B 5/0022; A61B 8/565; A61B 8/4427; A61B 8/13
USPC 600/301; 600/440; 600/382

(57) ABSTRACT

A portable device utilizes an ultrasound probe, which is placed on the abdomen of a mother and allows ultrasound video and pictures, as well as Doppler heartbeat detection of the fetus. It is also capable of recording fetal brain activity induced by constant-wave or pulsed-wave ultrasound stimuli; and analyzing the fetal EEG measurements. The electrical brain waves are detected by a sensor, amplified, digitized, and analyzed in one portable device, using analog and/or digital filters to improve the signal/noise ratio. The portable computer uses augmented reality imaging and quantitative EEG analysis software to compare the data from the fetus to normative data or to prior states of the fetus' own data. The EEG signals are recorded for an extended period of time and can be analyzed in real time or at a later time for rhythmicity patterns indicative of epilepsy or other developmental brain disorders.

This application is a continuation-in-part of U.S. Ser. No. 13/396,233 in the name of the inventor Marianna Kiraly filed on Feb. 14, 2012, now pending, which in turn claims the priority of Provisional Patent Application Ser. No. 61/627,626, filed on Oct. 14, 2011, in the name of the inventor Marianna Kiraly.

### FIELD OF THE INVENTION

The present invention relates to basic developmental neuroscience, portable medical obstetric procedures and devices, and more particularly to the non-invasive monitoring of the brain physiology states of a developing human embryo inside the mother's womb.

### BACKGROUND OF THE INVENTION

Many of the developmental disorders of childhood—cerebral palsy, epilepsy, cognitive impairment from prematurity and autism—appear to result from an interaction of complex genetic traits and environmental factors. Likewise, adult psychiatric diseases may have their origins in impaired early, even fetal development, as proposed for schizophrenia [1]. Despite major efforts, these prevalent, debilitating, life-long disorders remain biologically unexplained. Based on animal studies, the development of most types of epilepsy, cerebral palsy, autism and schizophrenia is suggested to link to neonatal seizures and various disturbances during embryogenesis. The intimate connection between mother, fetus and placenta, the vast array of neuroactive hormones expressed in the mother or in the placenta, or a variety of other environmental factors (injuries, drug treatments, immune responses, infections, hypoxic stress) make the targets when investigating fetal environmental disruptions that can affect the brain.

Perhaps one of the most important and challenging neuroscience research tasks would be to study the role of early brain activity in developmental plasticity and in the activity-dependent formation of neural circuits. It is evident that before birth, the immature brain expresses primitive, self-balancing rhythmic activity—with no complex excitatory and inhibitory synapses—in order to protect the developing brain from uncoordinated network activities or hyperexcitation. This early electrographic pattern was recorded in preterm human infants and newborn animals during sleep, immobility or feeding behavior and contains long silence periods. Although it is poor in information content, and it is not necessarily associated with any specific information—e.g. presence in the retina before eye opening—, but still, it is indispensable to turn on the machine and ignite the network [2].

In previous human studies (for review see [3]) it was noted that the cortical EEG (electroencephalogram) recorded in neonates during the second half of gestation is organized in intermittent bursts that are separated by periods of virtually complete suppression of activity that could last for minutes. With maturation, suppression of activity between the bursts becomes less pronounced. At full-term, some discontinuity is still evident. At mid-gestation, the activity is dominated by delta waves of 0.3-2.0 Hz. By the seventh month of gestation, slow oscillations are intermixed with rapid rhythms. Each event of rapid activity consists of 8-25 Hz spindle-like, rhythmic activity superimposed on 0.3-1.5 Hz delta waves. These rhythms (referred as “delta brushes”) are predominantly expressed in central areas before 28 weeks, and are then recorded in central, temporal and occipital areas from 28 weeks to near term. Presence of delta brushes in EEG from preterm infants serves as a criterion of normal development, whereas their absence is indicative of brain pathology and poor prognosis. In addition to delta brushes, several other patterns have been described in premature neonates.

In most cases, it is already too late after birth to permanently reverse the poor neurological outcomes, as they are being developed during embryogenesis due to microenvironmental alterations, which may change the process of neuronal migration and result in brain malformations or the establishment of incorrect or defective synaptic connections. Even though these specific, immature rhythmic activity patterns could be perfect indicators to identify each stage of the healthy functional brain development, it is very difficult to capture them due to lack of continuity or the movements of the fetus. Interestingly in the adult brain, focused ultrasound stimulation has been shown to both enhance and suppress spontaneous or electrically evoked field potentials; depending on its mode of operation (8-14). Therefore, non-invasive constant-wave (CW) and pulsed-wave Doppler (PW) ultrasound stimulation are powerful tools for generating fetal brain potentials, and use them for diagnostic purposes.

Recording of EEG signals is generally known in the medical arts. Use of ultrasound to display a fetus or to measure cerebral circulation by using the transcranial Doppler method is also generally known in the medical arts. Further, recording of fetal brain wave signals is known in the prior art, for example in U.S. Patent No. 20020193670, U.S. Patent No. 20100274145, U.S. Pat. No. 6,556,861 to Prichep, and in U.S. Pat. No. 7,016,722 to Prichep. The entire disclosures of these patents are expressly referred to and incorporated herein by reference thereto.

A flowchart of the device of U.S. Pat. No. 6,556,861 is shown herein as FIG. 1, which represents the prior art. The prior art steps shown in FIG. 1 are described in detail in that referenced patent specification, which as noted in the foregoing paragraph has been incorporated herein by reference.

It is a problem in the prior art to detect signs of epilepsy or other brain injuries or disorders in a developing fetus, as in most cases these are not correlated with responses to auditory stimuli. There is accordingly a need in the prior art for a non-invasive, safe brain stimulator that induces brain activity reproducibly, in different stages of the brain development.

It is a further problem and need in the prior art to provide a portable fetal-EEG recording device that is extremely sensitive, detecting potentials of even below 1-2 microvolts, capable of detecting and recording signals over an extended period of time, and perform the steps of analyzing the recorded signals for signs of developmental brain disorders in the developing fetus.

### SUMMARY OF THE INVENTION

In the present invention, the brain activity of the fetus is non-invasively induced by exposure to a series of constant-wave and/or pulsed-wave ultrasound irradiation treatment; and detected and analyzed in a portable fetal-EEG recording device. This is a procedure performed for an extended period of time using sensitive but comfortable, lightweight equipment (preferably a hand-held device). Recording fetal-EEG signals is of great importance, as these can serve as indicators for certain unhealthy conditions or environmental factors (e.g. altered maternal hormone levels, stress, drug treatment, etc.) that may risk the normal brain development of the fetus. The identification and exclusion of such factors and conditions during embryogenesis may help to avoid the development and progression of several neural disorders that are already untreatable after birth.

One or a grid of detecting sensor electrodes is removably attached to the abdominal skin of the pregnant woman, in close proximity to the head of the fetus. The electrical connectivity between the sensor and the abdominal skin can be improved by using an adhesive gel enriched with electrolytes. The visualization of the fetus can be performed using the “augmented reality” smartphone and tablet tool, for the user's convenience.

The sensor electrode connected to the fetal-EEG recording device is capable of detecting microvolt level fetal brain activity patterns, which can be recorded using similar low-noise ( 1/28l microvolt) amplification and optional bandpass filtering methods as known to be used for neurophysiology research purposes.

In order to better understand the measured data, an ultrasound probe (operated at 3.5-5 MHz frequency) can be connected to the “portable fetal-EEG recording device”, and the position of the fetus can be real-time monitored on the display of the device, in order to avoid the misinterpretation of data caused by movement of the fetus subsequent to the application of the electrodes resulting in incorrect readings, and which could therefore cause certain movement artifacts. The same or another ultrasound probe (operated at 2-8 MHz) connected to the same device may serve as a Doppler heart monitor for the fetus. When placing one of the sensor electrodes in close proximity to the heart of the fetus, it may serve as an ECG electrode. The simultaneous use of the Doppler ultrasound probe and ECG electrode may help the user make sure that the operation mode of the device is correct and both of the methods work properly. Monitoring the fetal heart frequency may provide additional information about the current activity of the fetus (e.g. allow the determination of its awake and sleep states).

Further computational (software) filtration and analysis of all electrical recordings can be performed in accordance with those known from conventional routine clinical EEG-recording methods. This may help in identifying electrical artifacts, as well as noise derived from the heart or muscles of the fetus or mother (e.g. fetal eye-movements). The portable fetal-EEG recording device provides an output for an Internet connection, and therefore allows all of the recorded ultrasound images and videos, raw and analyzed EEG recordings to be broadcasted in real-time, or later shared with the obstetrician/gynecologist, pediatric neurologist or any friends or family members of the user.

### BRIEF DESCRIPTION OF THE DRAWINGS

FIG. 1 shows a flowchart for a prior art device according to U.S. Patent No. 6,556,861, and is described therein.

FIG. 2 is a simple schematic demonstration of the noninvasive fetal-EEG device, ultrasound module, and electrodes according to the present invention.

FIG. 3 is a flowchart depicting steps in the use of device and method according to the present invention.

FIG. 4 is a schematic view of a portable device according to the present invention, showing structural features thereof and connections with an ultrasound device and a fetal-EEG recording device.

FIG. 5 is a schematic view of a portable device according to the present invention, showing functional connections and output features thereof, as well as connections with an ultrasound device, a fetal Doppler signal detecting device, and a fetal-EEG recording device.

FIG. 6 is a schematic flowchart of steps showing use of the invention of FIGS. 2-5.

### DETAILED DESCRIPTION OF THE INVENTION AND METHOD

The present invention, discussed in detail hereunder, relates to device and a method for using the device to induce fetal EEG signals by exposing the fetus to constant-wave or pulsed-wave brain stimulation, and to detect signs of normal and abnormal embryonic development. The device of the present invention provides an Internet connection, and it serves as an apparatus for performing and analyzing fetal-EEG and ECG recordings, ultrasound imaging and Doppler heartbeat detection.

Technologies which can be used in the present invention and which are commercially known and available for use, are known in the art and samples of these are as follows. The type of electrodes and method of use feasible for the present invention are known, for example in U.S. Pat. No. 6,162,101 issued on Sep. 3, 1998 to Fisher and Iversen; U.S. Pat. No. 6,024,702 issued on Feb. 3, 1997 to Iversen; U.S. Pat. No. 5,961,909 issued on Sep. 3, 1997 to Iverson; U.S. Pat. No. 5,902,236 issued on Sep. 3, 1997 to Iversen; as well as in other patent documents. The possibility of recording electrical brain and heart activity of a fetus in utero has also been published [4, 5].

An abdominal ultrasound unit capable of performing advanced ultrasound measurements for obstetrical use is known and commercially available. Portable scalp-EEG recording instruments and portable Doppler devices capable of determining fetal cerebral circulation and heart rate have been commonly used and commercially available for a long time.

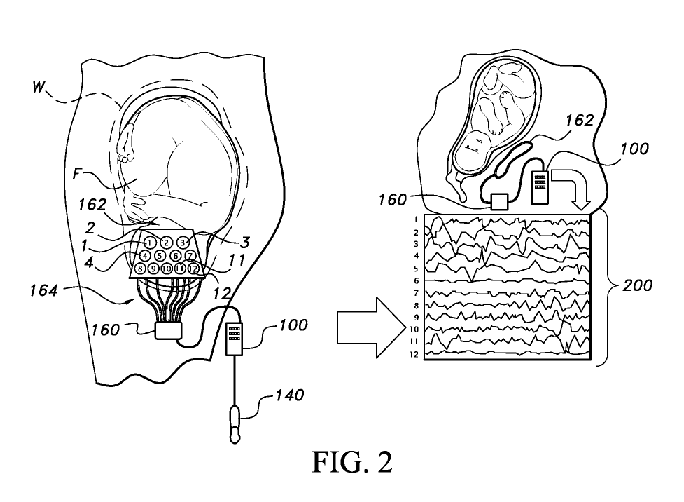

FIG. 2 is a simple schematic demonstration of a portable device 100, which is shown in FIG. 2 as being connected by any communication means including Bluetooth, Wireless Internet or wires to a fetal EEG detecting device 160 and to an ultrasound module 140. The fetal-EEG detecting device 160 is connected to an electrode or electrode sheet 162 by a plurality of connecting wires 164. The biosensor electrode or electrode sheet 162 has a plurality of sensitive electrodes thereon, of the type mentioned hereinabove, having a reference and multiple detector electrodes 1, 2, 3, 4, . . . , 11, and 12. The electrodes 1, 2, 3, 4, . . . , 11, and 12 can be numbered differently, and can be arranged in other types of geometrical locations, without departing from the scope of the present invention. The biosensor electrode or electrode sheet 162 is removably attached to the patient's skin; it may also use conductive gel, providing rapid attachment and acceptably low noise.

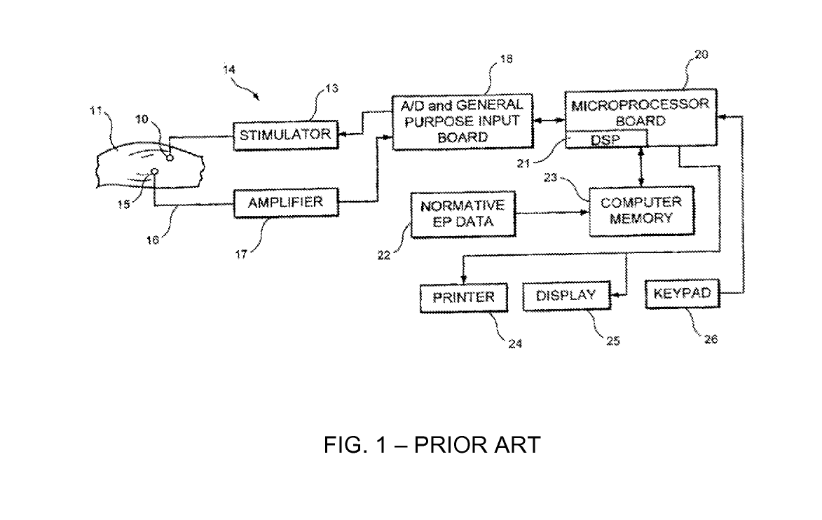

The ultrasound module 140 is an abdominal probe operated at 3.5-5 MHz in order to determine the position of the fetus, and is operated by a special imaging software capable of recording high-resolution videos and images, and can be any commercially available ultrasound device compatible with the present invention. It is also capable of Doppler fetal heartbeat and cerebral blood flow detection (operated at a range of 2-8 MHz). The fetal EEG detecting device 160 can be that shown in the above-mentioned prior art excluding the stimulator unit (FIG. 1), or can be a commercially available or custom developed device. These and other variations are all contemplated as being within the scope of the present invention.

In FIG. 2, the non-invasive fetal-EEG device is shown overlying a head and/or heart of a fetus F within a uterus W shown on the left hand side of FIG. 2. The right hand side of FIG. 2 depicts a schematic side view of the mother's uterus and the fetus, showing the electrode/electrode sheet 162 disposed on the mother's abdominal region directly over the head and/or heart of the fetus. Once the electrodes are positioned above the targeted area, ultrasound stimulation is directed to the head of the fetus using the constant-wave (imaging) or pulsed-wave (Doppler) mode of the ultrasound probe 140. The duration of the ultrasound exposure may vary, but should not exceed 30 minutes to avoid any possible disturbances in neuronal migration. The ultrasound probe is operated at a frequency range between 2 and 8 MHz, pulse-repetition frequency 1 Hz-2 kHz range. Illustrated output signals from the detector electrodes 1, 2, 3, 4, . . . , 11, and 12 are shown at the output graph 200. These brain waves and/or ECG signals are recorded at a preferred sampling frequency of minimum 4 kHz, digitized, amplified using high input impedance of at least 1 MegaOhm, low-noise ( 1/28l microvolt) amplification, and may preferably be filtered at a bandpass frequency of 1 to 64 Hz. The range of 1 to 64 Hz is typical, but not limiting; and a range up to 70 Hz is contemplated as being useable in the present invention. In addition, the recorded traces may also be integrated, in order to make the mathematical (statistical) analysis and peak-detection easier, and to provide a simple way for measuring wave amplitude, duration, integrated brain wave area, burst frequency or other quantitative parameters. The output graph 200 is by way of illustration only; in actual use a graph is not displayed but instead the signals are monitored and recorded continuously and over a relatively long period of time, e.g. hours or days. The signals can be processed either in real time or at a later time by specific recording and analysis software, and can be transmitted by the portable device 100 using the internet or using cell phone transmission, etc., to a computer for analyzing the signals, or to an obstetrician or other professional. Further software analysis and corrections provide additional noise reduction, and may help eliminate movement artifacts (i.e., where movement of the fetus occurs after placement of the electrodes resulting in incorrect output readings) or other non-specific signals (e.g. muscle activity, eye movement, mother's heart signals, etc.). The visualization of the fetus can be performed using the “augmented reality” feature in case if the recording device is smartphone or tablet device, for the user's convenience. Using the built-in camera of these portable computers and combining it with the image processing tools of the recording software, the real-time position of the fetus relative to the ultrasound probe can be projected to the mother's abdomen, on the computer screen. This feature helps the user attach the electrodes above the area of interest, which in this case is the head of the fetus.

The portable device 100 combined with the fetal EEG detecting device 160 and the ultrasound module 140, constitutes a small, compact and portable EEG monitoring system, which can make it possible for physicians to follow the maturation of fetal brain activity in a real-time manner during high-risk pregnancies, maternal infections, hypoxia, stress, or other conditions. Qualitative and quantitative data evaluation methods described in the prior art and studies [6, 7] can be applied to determine the functional developmental status of the fetus. The raw and analyzed ultrasound-induced and spontaneous fetal EEG data can be compared to reference spontaneous fetal EEG data from a control group to determine one of an abnormality and normality of the brain activity and heart rate of the fetus being monitored. However, in the lack of a proper instrument capable of inducing and detecting human fetal brain waves in utero, yet little is known about the brain activity of unborn human fetuses. Therefore this present invention will be a useful tool for scientific research purposes, in order to better address and understand the functional brain development process of human embryos.

The small, portable EEG-device 100 of the present invention is capable of recording data all day long, causing no inconvenience in continuing the usual activities of the user's everyday life. The registered waves can be analyzed either real-time, or later in the office of a gynecologist or pediatric neurologist. This technology can be applied in construction of the portable device 100 of the present invention, which is thereby made as a small, user-friendly and affordable fetal-EEG device specifically designed for clinical purposes, which will be ideal for everyday usage and reliable diagnostics.

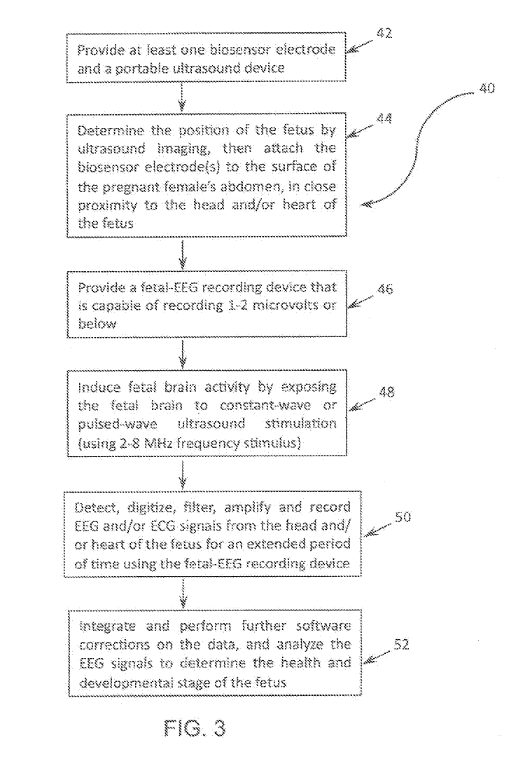

FIG. 3 is a flowchart 40 depicting steps 42, 44, 46, 48, 50 and 52 in the use of the device and method according to the present invention.

The steps include (step 42) providing at least one biosensor electrode and a portable ultrasound device, or—where the biosensor electrode is replaced by an electrode grid or sheet—providing an electrode grid or sheet and a portable ultrasound device, then determining the position of the fetus (step 44) by ultrasound imaging. Step 44 includes attaching the sensor or electrode sheet having the EEG electrodes to the surface of the abdomen right above the head and/or heart of the fetus (e.g., in close proximity to the head and/or heart of the fetus).

Following the above steps 42 and 44, further providing (step 46) a portable fetal-EEG recording device (such as the portable device 100 described hereinabove with reference to FIG. 2) that is extremely sensitive, detecting potentials of 1-2 microvolts or below. The next step (step 48) is to induce fetal brain activity by exposing the fetal brain to constant-wave or pulsed wave ultrasound stimulation (using 2-8 MHz frequency stimulus). This step is followed by step 50 to detect, digitize, filter, amplify and record EEG and/or ECG signals from the head and/or heart of the fetus for an extended period of time using the portable fetal-EEG recording device. It is recommended to repeatedly determine the position of the fetus by ultrasound imaging, in order to determine the movement of the fetus between the time of application of the electrodes to the time of later measurements. This is intended to prevent occurrence of artifacts in the recordings.

As noted above, the above steps 42, 44 and 46 are followed by the fetal ultrasound-stimulation (step 48). At step 48, fetal brain activity is evoked by directing the constant-wave or pulsed-wave ultrasound probe (2-8 MHz, 1 Hz-2 kHz pulse-repetition frequency in case if pulsed stimulation is applied) to the head of the fetus. The duration of the exposure may vary, but not exceed 30 minutes to avoid any disturbances in nerve cell migration. In some cases, even a combination of stimuli can be delivered to the fetal brain, and data of each response should be collected for analysis.

Following the above steps 42, 44, 46, 48, and 50, the method of the present invention includes further analyzing the result of step 50 (step 52). The purpose of step 52 is to integrate and perform further software corrections on the data, and to analyze the EEG signals to determine the health and developmental stage of the fetus. More specifically, in step 52 the recorded fetal-EEG signals are further analyzed for signs of neural network activity patterns indicative of brain disorders, and such analysis can include the steps of digitizing the signals, filtering the signals from all non-specific noise, amplifying the signals, integrating the signals, and storing the signals in a relatively small portable storage medium and/or an internet connection.

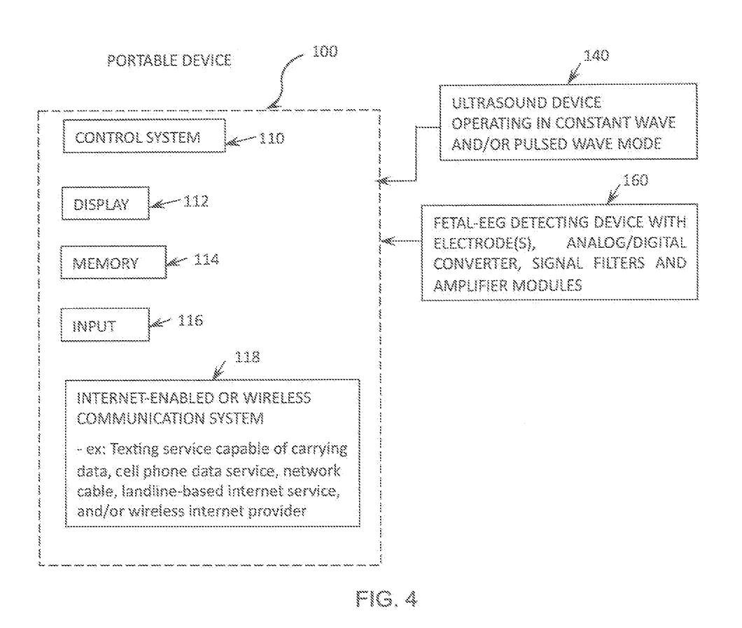

FIG. 4 is a schematic view of the portable device 100 according to the present invention, showing structural features thereof and connections with an ultrasound device 140 and a fetal-EEG recording device 160.

As seen in FIG. 4, the portable device 100 is shown in dashed outline, and preferably includes a control system 110, a display 112, a memory 114, an input means 116 (such as a touch pad, a keyboard, a mouse, or other input devices), and an internet-enabled or wireless communication system 118. The internet-enabled or wireless communication system 118 can be of a type already known in smartphone technologies, or it can be a custom-built portable device within the ambit of skill of any one having skill in the smartphone arts. The elements 110, 112, 114, and 116 can all be types which are present in existing smartphone technologies, or can be custom made within the ambit of skill of any one having skill in the smartphone arts and/or the smartphone application programming arts, utilizing the “augmented reality” feature for imaging and visualization.

As shown in FIG. 4, the device 100 is connected to the ultrasound device 140. Here, the ultrasound device 140 is operating in constant wave and/or pulsed wave mode. Further, the device 100 is connected to the fetal-EEG detecting device 160 which has one or more electrodes, an analog/digital converter, signal filters and amplifier modules.

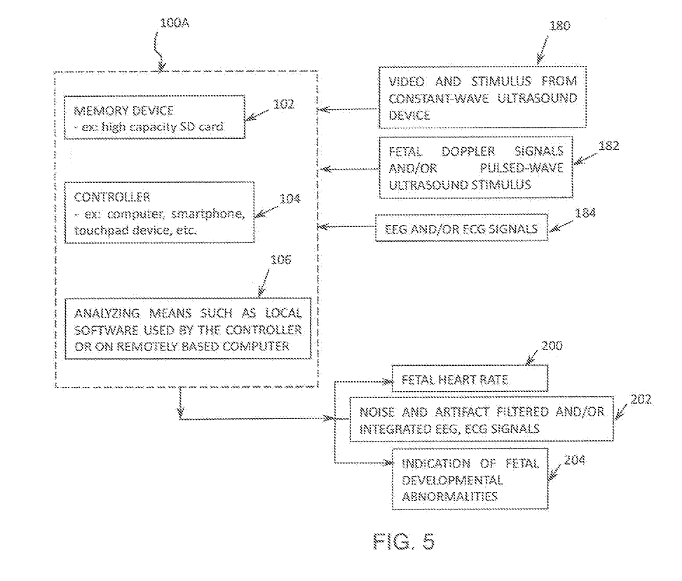

FIG. 5 is a schematic view of a portable device 100A according to the present invention, showing functional connections and output features thereof, as well as connections with an ultrasound device 180, a fetal Doppler signals and/or pulsed-wave ultrasound stimulus device 182, and a recording device 184 which records EEG and/or ECG signals. The portable device 100A can be similar or identical to the portable device 100 shown and discussed hereinabove, or it can be a variation of that device.

The portable device 100A includes a memory device 102 which can, for example, be a high capacity SD card or other type of memory device. The portable device 100A also includes a controller 104 which can, for example, be a computer or computer chip, a smartphone, smart touchpad device having computer techology, etc. Controllers are well known in the electronics arts, and have many types of variations and features; the present invention is not limited to any specific type of controller.

The portable device 100A also includes an analyzing function means 106 such as local software used by the controller 104, or else supplies data to a remotely based computer for software analysis using the internet or cell phone technology.

The portable device 100A provides outputs, which can include fetal heart rate 200, noise and artifact filtered stimulated or spontaneous EEG, ECG and/or integrated EEG signals 202, and an indication of fetal developmental abnormalities such as intrauterine seizures or other abnormal brain activity 204. These signals can be obtained using the software, and the detection and determination of normal and abnormal human fetal brain activity is an evolving field. It is anticipated that future discoveries may be made in this evolving field, and it is contemplated that the results of such discoveries can be used in the indication of abnormal fetal development 204.

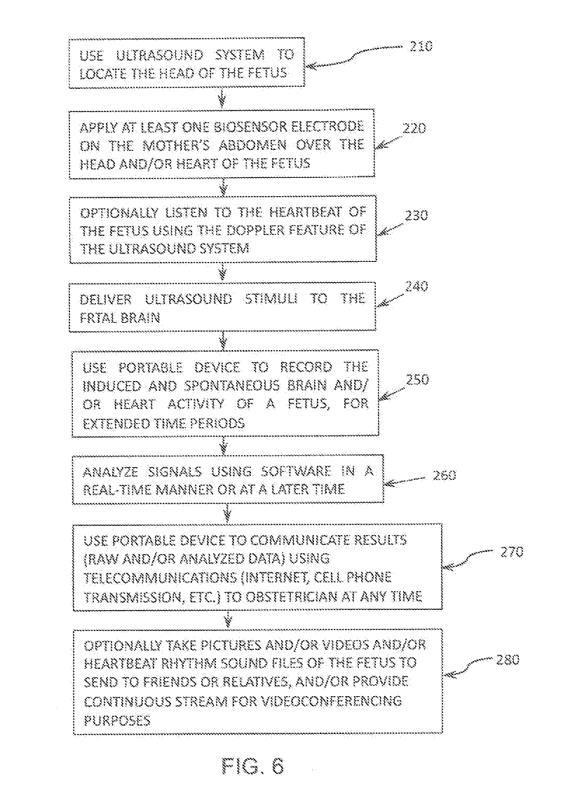

FIG. 6 is a schematic flowchart of steps showing use of the invention of FIGS. 2-5. Here, step 210 is use of the ultrasound system 180 to locate the head of the fetus. This can be done using the “augmented reality” feature of the imaging software. Then at step 220, the electrode sheet 162 is applied to the mother's abdominal region over the head and/or heart of the fetus. Step 230 is optional, and includes listening to the heartbeat of the fetus using the Doppler feature of the fetal Doppler signals from the ultrasound device 182. Step 240 is the step of delivering the ultrasound stimulus to the fetal brain with the constant-wave ultrasound system 180 or the pulsed-wave ultrasound system 182. Step 250 is using the portable device 100 or 100A to record the induced and spontaneous brain activity and/or the heart activity of the fetus (using the signals received from the electrode or electrode sheet 162) for extended time periods.

Also in FIG. 6, the step 260 is the step of analyzing the above-mentioned detected signals using software in a real time manner or at a later time. Step 260 is performed using the portable device 100 or 100A to communicate results (raw and/or analyzed data) using telecommunication means as discussed hereinabove (e.g. internet, cell phone transmissions, etc.) to an obstetrician or other professionals at any time. The Step 260 also is contemplated to include transmitting stored data saved over a relatively long period of time, and having that data analyzed by remote software, by an obstetrician, or by other professionals at any time.

Lastly, the optional step 270 is the step of using the ultrasound device 180 to take pictures and/or videos and/or sound files of the baby to send to relatives, friends, and/or medical professionals, and/or to provide a continuous stream of video for webcam or videoconferencing purposes.

### Additional Features

The present invention in contemplated as further including additional features. One such additional feature is a function of the imaging software that transforms the ultrasound images in order to make the electrode positioning easier. Such image transforming software would be within the ambit of any one having skill in the art of medical ultrasound imaging software. Originally, fetal ultrasound generates images of 45-90 degrees, kind of like virtual “sections”—these need to be rotated and projected to the abdominal plane. The software is also contemplated to utilize automatic image processing (e.g. head-shape recognition) to calculate and determine the proper positions where the electrodes need to be placed. This too would be within the ambit of skill of any one having skill in the art of medical ultrasound imaging software.

Another feature relates to future disease diagnosis. Typically, maternal blood is collected during pregnancy, in addition to umbilical chord blood and placenta samples during delivery. Comparison of the concentrations certain chemical compounds (e.g. hormones, enzymes, immune proteins) in certain patient groups in correlation with the fetal-EEG patters may propose novel prenatal therapies. For example, if certain abnormal fetal brain wave patterns can be correlated to high maternal blood component levels, then they may receive injections during pregnancy to prevent their fetus from developing certain neurological diseases.

The invention being thus described, it will be evident that the same may be varied in many ways. Such variations are not to be regarded as a departure from the spirit and scope of the invention and all such modifications are intended to be included within the scope of the claims.

### REFERENCES

1. Meyer U, Yee B K, Feldon J.: The neurodevelopmental impact of prenatal infections at different times of pregnancy: the earlier the worse? Neuroscientist. 2007 June; 13(3):241-56.
2. Ben-Ari Y, Gaiarsa J L, Tyzio R, Khazipov R: GABA: a pioneer transmitter that excites immature neurons and generates primitive oscillations. Physiol Rev. 2007 October; 87(4):1215-84.
3. Khazipov R, Luhmann N J. Early patterns of electrical activity in the developing cerebral cortex of humans and rodents. Trends Neurosci. 2006 July; 29(7):414-8. Epub 2006 May 19.
4. Khandoker, A. H.; Kimura, Y.; Palaniswami, M.; Marusic S.: Identifying fetal heart anomalies using fetal ECG and Doppler cardiogram signals. Computing in Cardiology, 2010 September; 891-4.
5. Lindsley D B.: Heart and brain potentials of human fetuses in utero. Am J. Psychol. 1987 Fall-Winter; 100(3-4): 641-6.
6. Scher M S, Turnbull J, Loparo K, Johnson M W.: Automated state analyses: proposed applications to neonatal neurointensive care. J Clin Neurophysiol. 2005 August; 22(4):256-70.
7. Vanhatalo S, Kaila K.: Development of neonatal EEG activity: from phenomenology to physiology. Semin Fetal Neonatal Med. 2006 December; 11(6):471-8. Epub 2006 October 2.
8. Legon W, Rowlands A, Opitz A, Sato T F, & Tyler W J (2012) Pulsed ultrasound differentially stimulates somatosensory circuits in humans as indicated by EEG and FMRI. (Translated from eng) PLoS One 7(12):e51177 (in eng).
9. Tufail Y, Yoshihiro A, Pati S, Li M M, & Tyler W J (2011) Ultrasonic neuromodulation by brain stimulation with transcranial ultrasound. (Translated from eng) Nat Protoc 6(9):1453-1470 (in eng).
10. Tufail Y, et al. (2010) Transcranial pulsed ultrasound stimulates intact brain circuits. (Translated from eng) Neuron 66(5):681-694 (in eng).
11. Tyler W J (2011) Noninvasive neuromodulation with ultrasound? A continuum mechanics hypothesis. (Translated from eng) Neuroscientist 17(1):25-36 (in eng).
12. Tyler W J, et al. (2008) Remote excitation of neuronal circuits using low-intensity, low-frequency ultrasound. (Translated from eng) PLoS One 3(10):e3511 (in eng).
13. Yoo S S, et al. (2011) Focused ultrasound modulates region-specific brain activity. (Translated from eng) Neuroimage 56(3):1267-1275 (in eng).
14. Rinaldi P C, Jones J P, Reines F, & Price L R (1991) Modification by focused ultrasound pulses of electrically evoked responses from an in vitro hippocampal preparation. (Translated from eng) Brain Res 558(1):36-42 (in eng).

What is claimed is:

1. A non-invasive method of inducing and recording electrical brain activity of a fetus in utero using constant-wave or pulsed-wave (Doppler) ultrasound, comprising the steps of:
(a) identifying the head of the fetus using an abdominal ultrasound probe connected to a portable or non-portable fetal-EEG recording device, by real-time observation of the ultrasound images and videos displayed by the device;
(b) removably connecting at least two sensor electrodes to the mother's abdomen above the head of the fetus to detect brain activity in the fetus, said at least two sensor electrodes producing output EEG signals;
(c) providing a portable fetal-EEG recording device that is extremely sensitive and capable of detecting potentials of 1-2 microvolts or below;
(d) inducing fetal brain activity by exposing the head of the fetus to one of constant-wave ultrasound stimuli and pulsed-wave ultrasound stimuli;
(e) detecting the EEG signals output from the at least two sensor electrodes from the head of the fetus for an extended period of time using the portable fetal-EEG recording device;
(e) analyzing the recorded fetal-EEG signals for network activity patterns indicative of epilepsy or other developmental brain disorders; and
(f) displaying the raw signals and the results of the analysis.

2. The method as claimed in claim 1, wherein in step (e) further including the steps of: digitizing the signals, optionally filtering the signals from non-specific noise, amplifying the signals, and storing the signals in a relatively small portable storage medium capable of connecting to the internet.

3. The method of recording as claimed in claim 1, further comprising the step of integrating the signal using a portable computer-based software or an integrator module.

4. The method of recording as claimed in claim 1, further comprising the step of using “augmented reality” visualization of the fetus by using the ultrasound probe or a detail on the probe as “interest point” or “fiduciary marker”, and projecting the ultrasound images relative to the position of the probe and the marker.

5. The method of recording as claimed in claim 1, further comprising the step of improving a signal to noise ratio of the recorded brain waves using portable computer-based analysis software.

6. The method of recording as claimed in claim 1, further comprising the step of displaying results of the analysis as an indication of the status of brain function of the fetus.

7. The method of recording as claimed in claim 1, wherein, in step (a), the Doppler ultrasound probe is used to produce an output representing fetal heart rate.

8. The method of recording as claimed in claim 1, further comprising the step of rotating, transforming and projecting the ultrasound images to the abdominal plane, adjusting their size to reflect the real size of the fetus proportional to the size of the ultrasound probe, and performing automatic fetal body part (e.g. head) recognition using an imaging software; in order to simplify and improve the accuracy of electrode positioning for the user.

9. The method of recording as claimed in claim 1, wherein in step (b), the at least two sensor electrodes are used produce an output representing a fetal electrocardiogram (ECG).

10. The method of recording as claimed in claim 1, further comprising the step of passing the recorded analog data through a band pass filter having a plurality of band pass frequency ranges within an overall frequency range of 1 to 64 Hz.

11. The method of recording as claimed in claim 1, further comprising the step of comparing the fetal EEG data to reference fetal EEG data from a control group or the same fetus' own previously recorded data to determine one of an abnormality and normality of the brain activity and heart rate of the fetus being monitored.

12. The method of recording as claimed in claim 1, further comprising the step of transmitting any of the images, videos or recordings via the internet.

13. A portable fetal-EEG recording device for recording ultrasound-induced EEG signals from a fetus in utero, comprising:
(a) an ultrasound probe connected to the portable fetal-EEG device, adapted to be placed on an abdomen of a mother of the fetus, to identify the position of the fetus and to localize its head;
and said ultrasound probe including computing means running an imaging software to real-time visualize the ultrasound images of the fetus;
(b) an ultrasound system to generate constant-wave or pulsed-wave ultrasound signals, or the combination these stimuli of 2-8 MHz frequency (pulse rate frequency: 1 Hz-2 kHz);
(c) at least two sensor electrodes adapted to be placed on the mother's abdomen for detecting electrical activity of the brain of a fetus;
(d) an amplifier-filter module connected to the at least two sensor electrodes to amplify the brain activity of the fetus detected by the at least two biosensor electrodes;
(e) an analog/digital converter converting the analog data to digital data;
(f) a portable computer-based quantitative analysis software capable of improving a signal to noise ratio of the digitized spontaneous brain activity data and analyzing the data;
(g) a display to real-time demonstrate the raw data and the results of the analysis as an indication of a status of the fetus;
(h) an imaging computer software capable of rotating, transforming and projecting the ultrasound images to the abdominal plane, adjust their size to reflect the real size of the fetus proportional to the size of the ultrasound probe, and performing automatic fetal body part (e.g. head) recognition; in order to improve the accuracy and simplify the electrode positioning for the user; and
(i) portable computer-based memory to store the data, and being capable of outputting said data for transmission to an external device or network.

14. The portable fetal-EEG recording device as claimed in claim 13, wherein the at least one sensor electrode includes an electrode grid, and one electrode in the grid is used as a reference electrode.

15. The portable fetal-EEG recording device as claimed in claim 13, wherein the computer system includes a filter having a plurality of band pass frequency ranges in an overall frequency range of 1 to 64 Hz.

16. The portable fetal-EEG recording device as in claimed in claim 13, wherein the amplifier-filter module (c) includes an integrator module.

17. The portable fetal-EEG recording device as claimed in claim 13, further comprising: an arrangement comparing the digitized fetal brain activity data to comparative digitized fetal brain activity data from a normal group of fetuses or the fetus' previously recorded own data to determine one of an abnormality and normality of the EEG/ECG signals.

18. A portable fetal-EEG recording device as claimed in claim 13, wherein said control system uses the fetal Doppler ultrasound signals to produce an output representing fetal heart rate.

19. A portable fetal-EEG recording device as claimed in claim 18, further comprising software used by said control system for analyzing the recorded EEG and ECG signals and producing an output representing the states of the fetus, namely sleep, awake or other states, and indicate any of fetal seizures and other abnormal brain activities.

20. The portable fetal-EEG recording device as claimed in claim 13; wherein:
the signals are monitored and recorded continuously and over a relatively long period of time, in a range from hours to days;
the portable device combined with the fetal EEG detecting device and the ultrasound module, constitutes a small, compact and portable EEG monitoring system, which can make it possible for physicians to follow the maturation of fetal brain activity in a real-time manner during high-risk pregnancies, maternal infections, hypoxia, stress, and other conditions; the raw and analyzed ultrasound-induced and spontaneous fetal EEG data can be compared to reference spontaneous fetal EEG data from a control group to determine one of an abnormality and normality of the brain activity and heart rate of the fetus being monitored; and
the portable device provides outputs which can include fetal heart rate, noise and artifact filtered stimulated or spontaneous EEG, ECG and/or integrated EEG signals, and an indication of fetal developmental abnormalities such as intrauterine seizures and other abnormal brain activity.
